(19) United States
(12) Patent Application Publication
(10) Pub. No.: US 2012/0310075 A1
(43) Pub. Date: Dec. 6, 2012

(54) MEDICAL MEASUREMENT SYSTEM AND METHOD

(75) Inventor: Trevor Glen Russell, Ferny Grove (AU)

(73) Assignee: THE UNIVERSITY OF QUEENSLAND, Queensland (AU)

(21) Appl. No.: 13/511,761

(22) PCT Filed: Nov. 26, 2010
(86) PCT No.: PCT/AU10/01591

§ 371 Date: Aug. 23, 2012

(30) Foreign Application Priority Data
Nov. 26, 2009 (AU) 2009905891

(51) Int. Cl. G06K 9/78; A61B 5/11

(52) U.S. Cl. 600/407; 382/106

(57) ABSTRACT

A method of measuring a range of movement of a joint of a patient including the steps of: capturing an image including at least part of the patient; processing measurement points positioned on the image; determining a measurement axis that connects the measurement points; determining a reference axis; and measuring an angle at an intersection of the measurement axis and the reference axis according to the range of movement of the joint of the patient. The invention also resides in a method of measuring a distance or displacement in an image including the steps of: providing a scale for the image; capturing the image including at least part of a patient and the scale; positioning a marker on the image that defines a distance to be measured; and measuring the distance using the marker and the scale.

### FIELD OF THE INVENTION

The present invention relates to a medical measurement system and method and more specifically to a system and method for measuring angles, angular displacement, linear distance and linear displacement and in particular a degree or a range of movement of a patient.

### BACKGROUND OF THE INVENTION

Clinicians, such as physiotherapists, traditionally use manual therapy and exercise to aid the recovery of a patient from an injury, illness and the like. In order to inform the diagnostic process and monitor the progress of the patient, the clinician may use clinical tools such as a goniometer to measure physical and movement parameters of the patient's body. Furthermore, measurements may be made with a clinical tape measure in order to determine heights, lengths or other physical attributes of the patient.

In order to make a measurement using clinical tools, the clinician identifies bony landmarks on the patient and aligns the clinical tool with the landmarks. This requires excellent observation and palpation skills together with a detailed knowledge of musculoskeletal anatomy. The landmark may be difficult to identify depending of the morphology of the patient and the measurement is therefore prone to errors.

Some of the problems associated with conventional measurement techniques using clinical tools are:
1) They are difficult to align with the patient;
2) The reference points for the measurement are not always readily identifiable;
3) There may be variations in measurements between clinicians;
4) A patient cannot use the clinical tool to make measurements on themselves;
5) There may be variations in the measurement depending on the make and model of clinical tool used; and
6) Clinical tools are not suitable for complex joint movements.

Additionally, measurements need to be manually recorded and analysed, which is a tedious process and subject to further error.

### OBJECT OF THE INVENTION

It is an object of the invention to overcome or alleviate one or more of the above disadvantages and/or to provide the consumer with a useful or commercial choice.

### SUMMARY OF THE INVENTION

In one form, although it need not be the only or indeed the broadest form, the invention resides in a method of determining a range of movement of a joint of a patient including the steps of:

capturing an image including at least part of the patient;

processing measurement points positioned on the image;

determining a measurement axis that connects the measurement points;

determining a reference axis; and

measuring an angle at an intersection of the measurement axis and the reference axis according to the range of movement of the joint of the patient.

Preferably, the method includes the step of displaying the angle on a display of a computer.

Suitably, the measurement axis and/or the reference axis are extrapolated such that the measurement axis and the reference axis intersect.

The reference axis may be a physical reference axis or a computer generated virtual reference axis.

Optionally, the virtual reference axis may be determined with respect to vertical using a device such as an accelerometer.

Preferably, the angle is recorded in a database on the computer with respect to time.

Suitably, successive angles are displayed to the clinician on the display of the computer to determine changes to the range of movement.

Suitably the image is captured from a photo or a video.

Preferably, the image is corrected for a parallax error.

Suitably, the parallax error is corrected using an algorithm based on the physical reference axis and the virtual reference axis and moves pixels in the image to create a corrected image.

Suitably the method includes a measurement of an object in the image according to a known measurement in the image and the object may include the patient.

Suitably, the measurement of the object is a linear distance.

Optionally, the known measurement is a physical object of known dimensions placed in the image. Alternatively, the known measurement is determined from two parallel coherent electromagnetic sources projected onto the object wherein the distance is known between the two parallel coherent electromagnetic sources. Optionally, the two parallel coherent electromagnetic sources are lasers or synchrotron radiation.

In another form the invention resides in a method of measuring a distance or a displacement in an image including the steps of:

providing a scale for the image;

capturing the image including at least part of a patient and the scale;

positioning a marker on the image that defines a distance to be measured; and

measuring the distance using the marker and the scale.

Preferably, the scale is provided by two or more parallel coherent electromagnetic sources projected onto the patient wherein the distance is known between the two parallel coherent electromagnetic sources.

Optionally, the two or more parallel coherent electromagnetic sources form two or more parallel lines on the image. Preferably the two or more parallel coherent electromagnetic sources are lasers.

Optionally, the scale is computer generated.

In another form, the invention resides in a system for determining a range of movement of a joint of a patient including;

a camera; and

a computer connected to the camera, the computer including

a processor; and

a memory coupled to the processor, wherein the memory includes computer readable program code components configured to cause:

capturing an image including at least part of the patient;

processing measurement points positioned on the image;

determining a measurement axis that connects the measurement points;

determining a reference axis; and

measuring an angle at an intersection of the measurement axis and the reference axis according to the range of movement of the joint of the patient.

Preferably, the measured angle is displayed on a display connected to the computer.

Preferably the measurement is written to a database stored on the computer. Suitably, the medical measurement system is accessed via the internet.

### BRIEF DESCRIPTION OF THE DRAWINGS

An embodiment of the invention, by way of example only, will be described with reference to the accompanying drawings in which:

FIG. 1 shows a block diagram of a medical measurement system according to an embodiment of the present invention;

FIG. 2 shows a diagram of a prior art method of measuring hip flexion of a patient;

FIG. 3 shows a flow chart of a method to determine a range of movement of a part of a body of a patient;

FIG. 4 shows an image of a patient on a treatment table to determine a hip flexion of the patient according to an embodiment of the present invention;

FIG. 5 shows an image of a patient on a treatment table to determine shoulder flexion of the patient according to a further embodiment of the present invention;

FIG. 6 shows an image of a patient on a treatment table to determine a cervical spine flexion of the patient according to a further embodiment of the present invention;

FIG. 7 shows an image of a patient on a treatment table to determine a cervical spine rotation of the patient according to yet a further embodiment of the present invention;

FIG. 8 shows an image of a patient on a treatment table to determine a forearm supination according to yet a further embodiment of the present invention;

FIG. 9 shows an image of a patient on a treatment table to determine a shoulder external rotation of the patient according to yet a further embodiment of the present invention;

FIG. 10 shows an image of a patient on a treatment table to determine a hip flexion of the patient according to an embodiment of the present invention;

FIG. 11 shows an image of a patient on a treatment table to determine a shoulder flexion of the patient according to an embodiment of the present invention;

FIG. 12 shows an image of a patient on a treatment table to determine a cervical flexion of the patient according to an embodiment of the present invention;

FIG. 13 shows an image of a patient on a treatment table to determine a cervical rotation of the patient according to an embodiment of the present invention;

FIG. 14 shows an image of a patient on a treatment table to determine a forearm supination of the patient according to an embodiment of the present invention;

FIG. 15 shows an image of a patient on a treatment table to determine a shoulder external rotation of the patient according to an embodiment of the present invention;

FIG. 16 shows an image of a patient to determine a foot length of the patient according to an embodiment of a further aspect of the present invention;

FIG. 17 shows an image of a patient to determine a height of a foot above the ground according to an embodiment of the present invention;

FIG. 18 shows an image of a patient including a scale in the form of a laser grid projected on to the image to compute a linear distance according to a further embodiment of the present invention;

FIG. 19 shows an image of a patient to determine a head measurement of the patient using the grid of FIG. 18 according to a further embodiment of the present invention;

FIG. 20 shows a diagram of a parallax error according to an embodiment of the present invention;

FIG. 21 shows a screen shot of a measurement of an angle between a patient's upper leg and lower leg with the camera perpendicular to the patient according to an embodiment of the present invention;

FIG. 22 shows a screen shot of the image of FIG. 21 where a camera is not perpendicular to a patient according to an embodiment of the present invention; and

FIG. 23 shows a screen shot of a corrected image of FIG. 22 according to an embodiment of the present invention.

### DETAILED DESCRIPTION OF THE PREFERRED EMBODIMENT

In this specification, adjectives such as first and second, left and right, and the like may be used solely to distinguish one element or action from another element or action without necessarily requiring or implying any actual such relationship or order. Words such as “comprises” or “includes” are intended to define a non-exclusive inclusion, such that a process, method, article, or apparatus that comprises a list of elements does not include only those elements but may include other elements not expressly listed, including elements that are inherent to such a process, method, article, or apparatus.

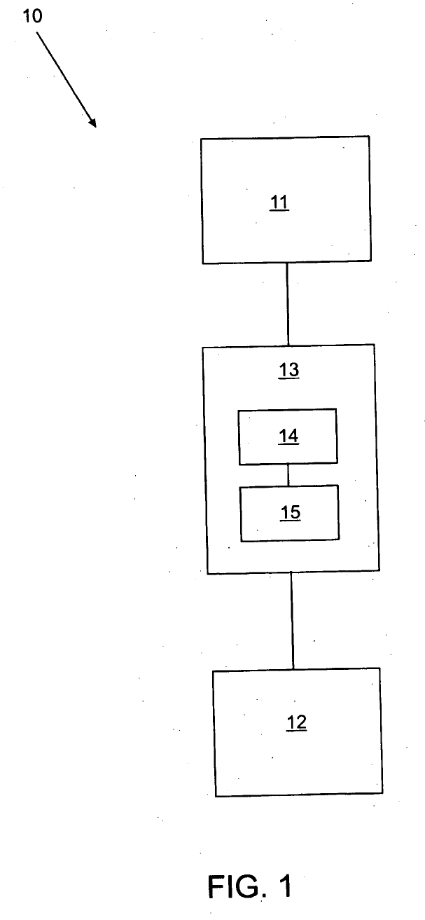

FIG. 1 shows a block diagram of a medical measurement system 10 according to an embodiment of the present invention. The system 10 includes a camera 11 and a display 12 connected to a computer 13. It should be appreciated that other elements may be connected to the computer 13 such as a keyboard and mouse. Furthermore, the computer 13 may be connected to the internet and the system 10 controlled remotely controlled via the internet.

The computer 13 includes a processor 14 and computer readable program code components in the form of software installed on a memory 15, for example on a hard disk, a random access memory (RAM), or any other applicable computer readable medium. The computer readable program code components are then processed by the processor 14 and are configured to cause execution of a method of the present invention.

The camera 11 may be separate to, or an integral part of the display 12 or computer 13 as would be understood by a person skilled in the art. The camera 11 captures one or more images, for example a photo or a video, that includes at least part of a patient performing a movement or posture test in order to make a measurement of a range or a degree of movement or a posture of the patient. Furthermore, dimensions such as linear distance of objects including the patient may be measured from the image. The images are transmitted to the computer 13 and analysed by the processor 14 in accordance with software installed on the memory 15.

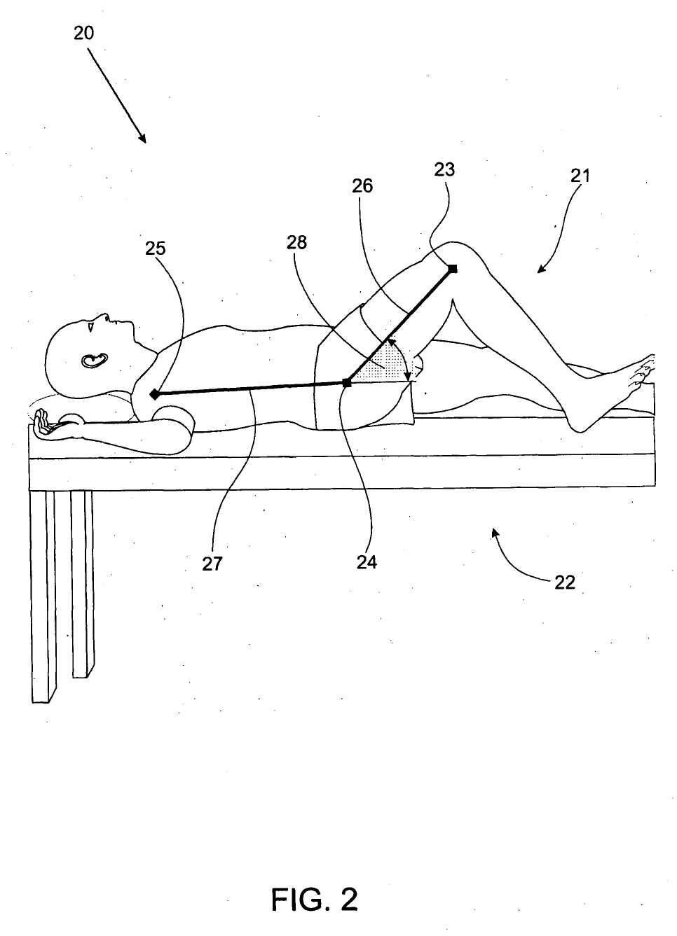

FIG. 2 shows a diagram 20 of a prior art method of determining a range of movement of a joint of a patient 21, in particular measuring hip flexion of the patient 21. The patient 21 is positioned on a treatment table 22 and a clinician locates bony landmarks on the body of the patient 21. In this example the clinician identifies the following bony landmarks; a distal point 23 or Lateral Condyle of the knee, a point of rotation 24 or Greater Trachanter and a proximal point 25 or a midline of a trunk of the patient 21.

Using a goniometer (not shown), the clinician aligns the goniometer with imaginary lines 26, 27 that join or connect the bony landmarks 23, 24, 25 in order to measure an angle 28 according to hip flexion.

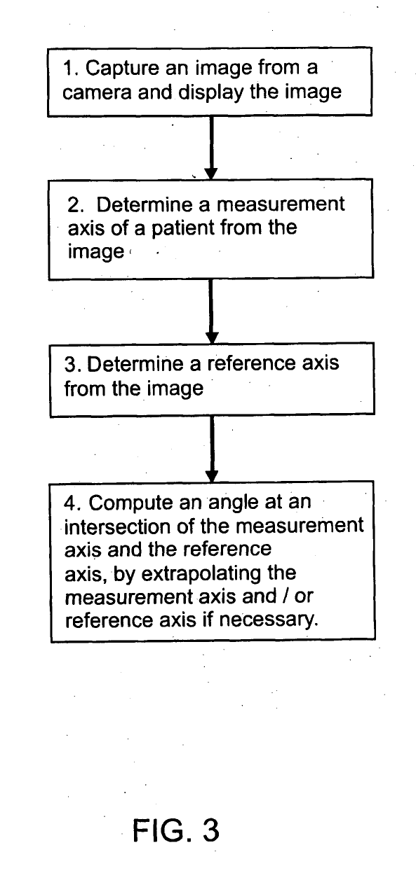

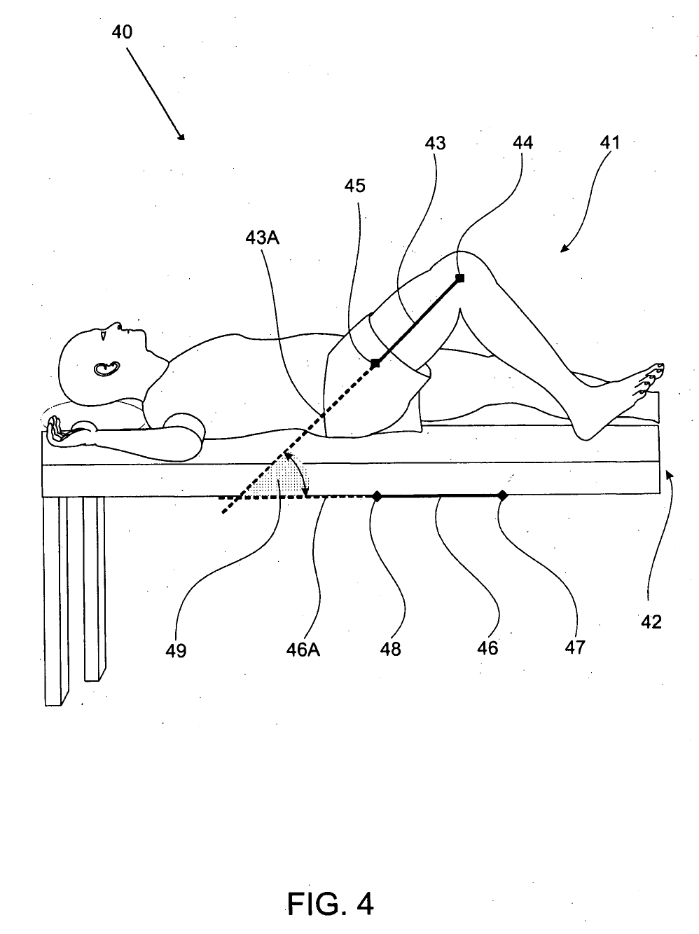

FIG. 3 shows a flow chart of a method to determine a degree or a range of movement of a part of a body and in particular a joint of a patient for the embodiments described in FIGS. 4 to 15. FIG. 4 shows an image 40 of a patient 41 on a treatment table 42 to determine hip flexion of the patient 41 according to an embodiment of the present invention. In this embodiment, the patient is lying down on the treatment table 42. Referring to FIGS. 3 and 4, at step 1 the image 40 is taken from a side of the patient 41 by the camera 11 and the image 40 is displayed to the clinician on the display 12 of the computer 13.

At step 2, the clinician may identify or determine a measurement axis 43 from the image 40 by positioning two measurement points 44, 45 on the image 40. Measurement point 44 corresponds with the distal point or Lateral condyle of the knee and measurement point 45 corresponds with a point along the shaft of the femur. The measurement points 44, 45 are processed by the computer 13 to determine the measurement axis 43 which connects measurement points 44, 45.

At step 3, the clinician may identify or determine a reference axis in the form of a physical reference axis 46 from the image 40 by positioning two reference points 47, 48 on the image 40 according to a horizontal support of the treatment table. The reference points 47, 48 are processed by the computer 13 to determine the physical reference axis 46 which connects reference points 47, 48. At step 4, an angle 49 is computed by the computer 13 at an intersection between the measurement axis 43 and the physical reference axis 46 according to hip flexion. It should be noted that the physical reference axis 46 is non-parallel to the measurement axis 43 in order that they intersect. In the present example the measurement axis 43 and the physical reference axis 46 do not intersect, thus the measurement axis 43 and the physical reference axis 46 are extrapolated. Extrapolated measurement axis 43A and extrapolated physical reference axis 46A are computed by the computer 13 with the aid of an algorithm. It should be noted that the measurement axis 43 and the physical reference axis 46 may not need to be extrapolated depending on their position relative to each other. For example if the physical reference axis is a vertical door frame positioned directly behind the measurement axis, neither the physical reference axis nor the measurement axis requires extrapolating.

It should also be appreciated that the physical reference axis may be a window frame, a leg of the treatment table 42 or any other suitable fixed point of reference that has a known orientation with respect to a vertical plane, i.e. with respect to the direction of action of gravity. It should be appreciated that the reference axis may be normal to the vertical plane; i.e. the reference axis may be a horizontal plane.

Furthermore, the computer 13 may compute a reciprocal angle of the measured angle 49, depending on the reference axis and the measurement axis. The reciprocal angle may be + or −90 degrees or + or −180 degrees of the measured angle.

Although FIG. 4 shows an image of the whole patient 41, it should be appreciated that that the image may include only part of the patient 41 according to the part of the body or joint being measured. For example to measure hip flexion the image only needs to show an upper leg of the patient 41.

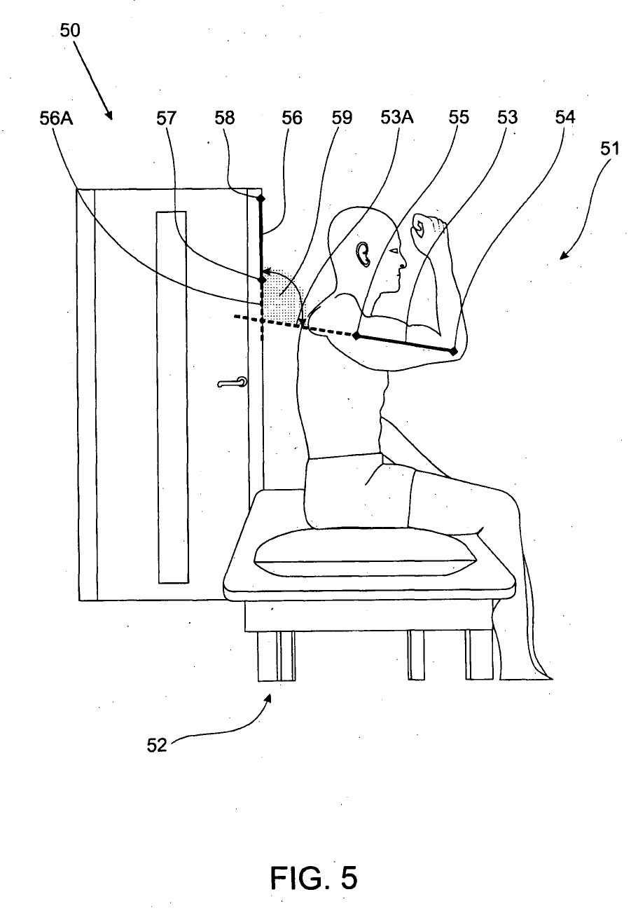

FIG. 5 shows an image 50 of a patient 51 on a treatment table 52 to determine a shoulder flexion range of motion of the patient 51 according to a further embodiment of the present invention. In this embodiment, the patient 51 is sitting on the treatment table 52 with his or her legs hanging over the side of the treatment table 52. Similarly to the first embodiment, the image 50 is taken from a side of the patient 51 by the camera 11 and displayed to the clinician on the display 12 of the computer 13.

The clinician may identify a measurement axis 53 by positioning two measurement points 54, 55 corresponding to the lateral epicondyle of the elbow and a point along the humerous of the patient 52. The measurement points 54, 55 are processed by the computer 13 to determine the measurement axis 53 which connects measurement points 54, 55. A physical reference axis 56 may then be identified by positioning two reference points 57, 58 on the image 50 according to a door frame. The reference points 57, 58 are processed by the computer 13 to determine the physical reference axis 56 which connects reference points 57, 58. The computer 13 may compute shoulder flexion by measuring an angle 59 at an intersection of an extrapolated measurement axis 53A and an extrapolated physical reference axis 56A. Again, it should be appreciated that if the door frame was directly behind the patient, the measurement axis 53 would intersect with the physical reference axis 56 without the need to extrapolate the measurement axis 53 or the physical reference axis 56. It should also be appreciated that the measurement axis and the physical reference axis may be extrapolated in either direction.

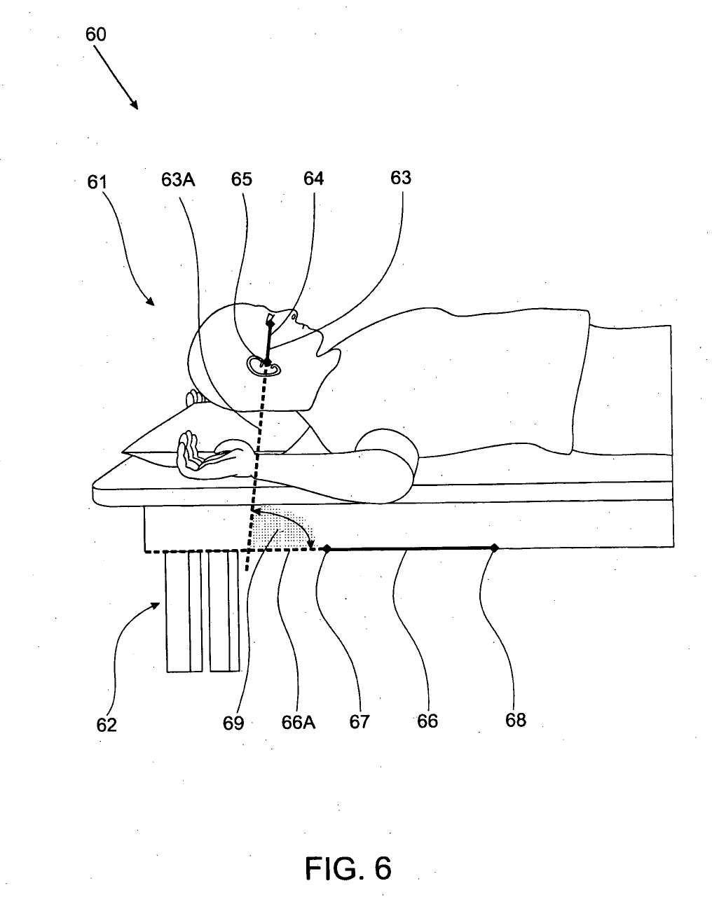

FIG. 6 shows an image 60 of a patient 61 on a treatment table 62 to determine the cervical spine flexion of the patient 61 according to further embodiment of the present invention. In this embodiment the patient 61 is lying on the treatment table 62 and the image 60 is taken from a side of the patient 61. A measurement axis 63 is determined from measurement points 64, 65 positioned on the patient's lateral canthus of the eye 64 and the tragus 65 of the patient's 61 ear. The measurement points 64, 65 are processed by the computer 13 to determine the measurement axis 63 which connects measurement points 64, 65. A physical reference axis 66 is determined from reference points 67, 68 positioned on a frame of the treatment table 62. The reference points 67, 68 are processed by the computer 13 to determine the physical reference axis 66 which connects reference points 67, 68. An angle 69 according to cervical spine flexion is computed from an intersection of an extrapolated measurement axis 63A and an extrapolated physical reference axis 66A. Again it should be appreciated that the angle may be computed from a reciprocal of the measured angle 69.

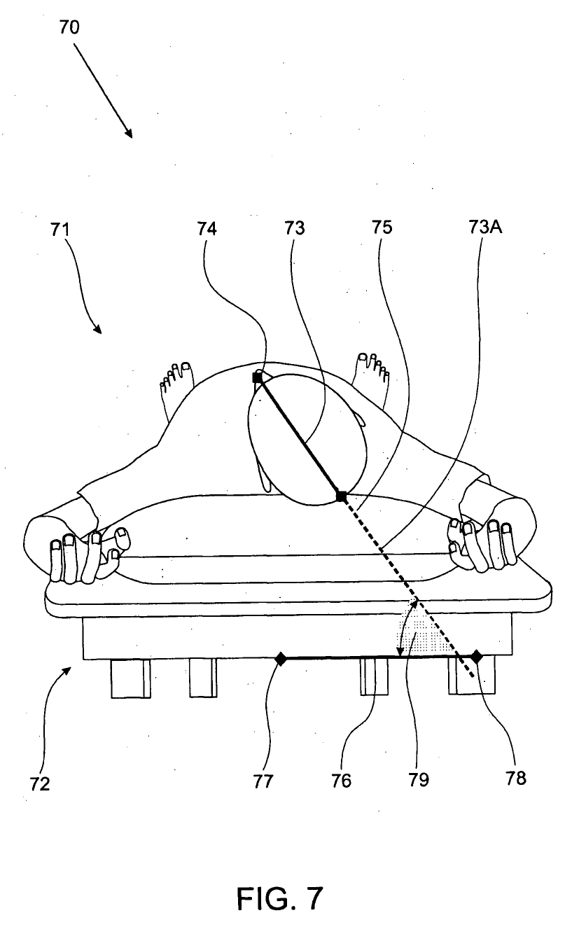

FIG. 7 shows an image 70 of a patient 71 on a treatment table 72 to determine a cervical spine rotation of the patient 71 according to yet a further embodiment of the present invention. In this embodiment the image 70 is taken from a top of the patient 71. A measurement axis 73 is determined from measurement points 74, 75 positioned on a tip of the patient's nose 74 and a mid point 75 of the back of the patient's 71 head. The measurement points 74, 75 are processed by the computer 13 to determine the measurement axis 73 which connects measurement points 74, 75. A physical reference axis 76 is determined from reference points 77, 78 positioned on a frame of the treatment table 72. The reference points 77, 78 are processed by the computer 13 to determine the physical reference axis 76 which connects reference points 77, 78. In the present embodiment, an angle 79 according to cervical spine rotation of the patent 71 is computed at an intersection between an extrapolated measurement axis 73A and the physical reference axis 76.

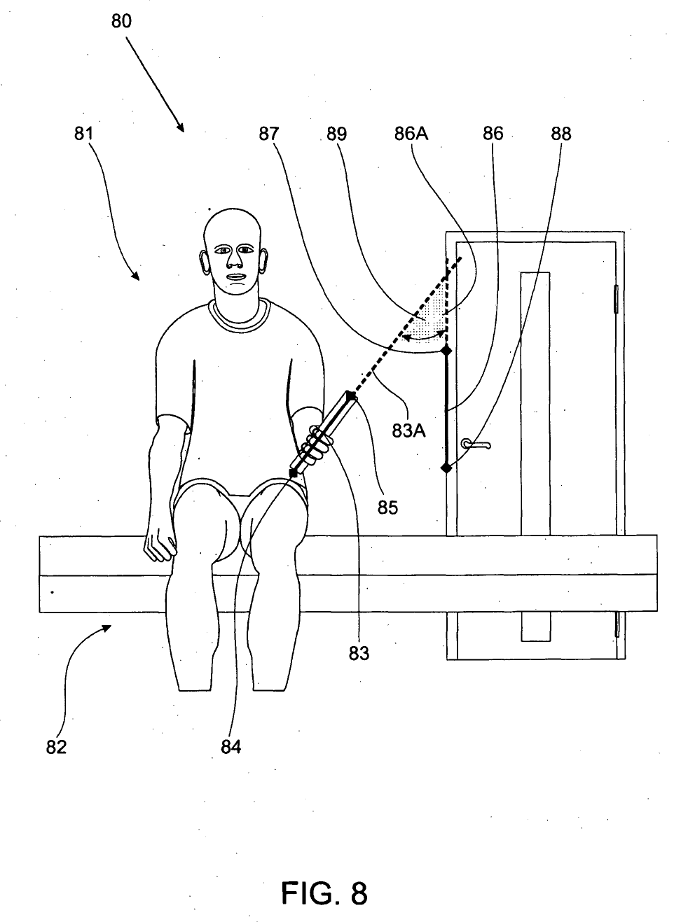

FIG. 8 shows an image 80 of a patient 81 on a treatment table 82 to determine a forearm supination according to yet a further embodiment of the present invention. In this embodiment the patient 81 is sitting on the treatment table 82 and the image 80 is taken from a front of the patient 81. A measurement axis 83 is determined from measurement points 84, 85 positioned on a straight edge held by the patient 81, for example a baton. The measurement points 84, 85 are processed by the computer 13 to determine the measurement axis 83 which connects measurement points 84, 85. The baton provides an easy point of reference for the clinician to identify the measurement axis 83. A physical reference axis 86 is determined from reference points 87, 88 positioned on a frame of a door. The reference points 87, 88 are processed by the computer 13 to determine the physical reference axis 86 which connects reference points 87, 88. An angle 89 according to forearm supination is computed at an intersection between an extrapolated measurement axis 83A and an extrapolated physical reference axis 86A.

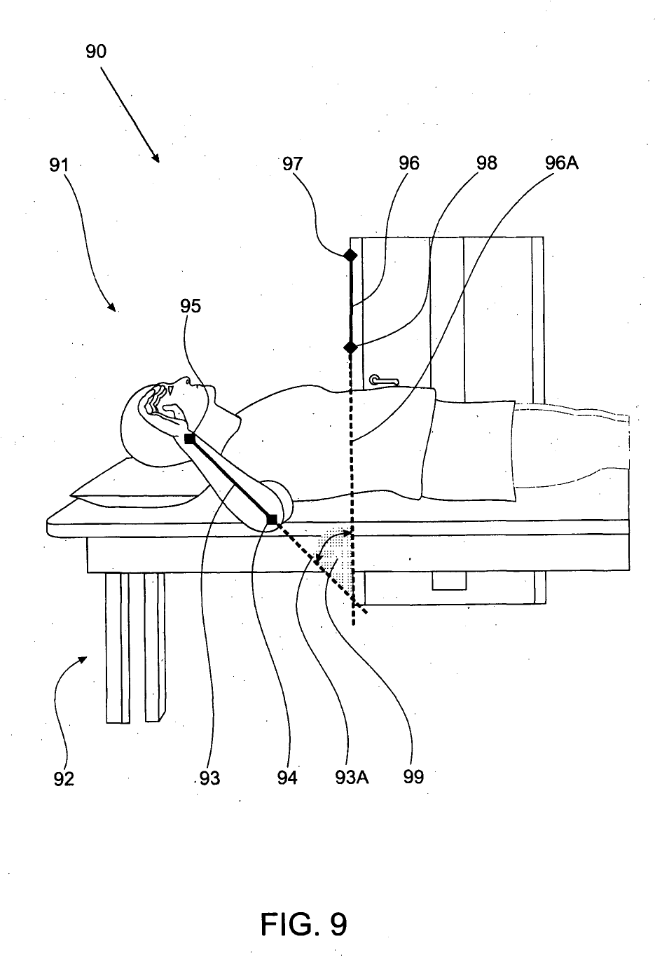

FIG. 9 shows an image 90 of a patient 91 on a treatment table 92 to determine a shoulder external rotation of the patient 91 according to yet a further embodiment of the present invention. In the present embodiment the patient 91 is lying on the treatment table 92 to determine a shoulder external rotation and the image 90 is taken from a side of the patient 91. A measurement axis 93 is determined from measurement points 94, 95 positioned on a forearm of the patient 91. The measurement points 94, 95 are processed by the computer 13 to determine the measurement axis 93 which connects measurement points 94, 95. A physical reference axis 96 is determined from reference points 97, 98 positioned on a frame of a door. The reference points 94, 95 are processed by the computer 13 to determine the physical reference axis 96 which connects reference points 97, 98. An angle 99 according to shoulder external rotation in computed at an intersection of an extrapolated measurement axis 93A and an extrapolated physical reference axis 96A.

Although the measurement axis and the reference axis have been determined by the clinician, it should be appreciated that the measurement axis and the reference axis may be determined by the computer 13 using feature recognition software.

In use, a clinician positions a patient substantially perpendicular or orthogonal to the camera's 11 field of view. Alternatively, the patient may be given instructions how to position themselves, for self-measurement systems and remotely controlled systems and systems controlled via the internet. An image or a video clip is taken by the camera 11 and sent to the computer 13 to determine an angle or a linear distance from the image. The clinician positions measurement points and reference points on the image or video clip that correspond to a measurement axis and a reference axis respectively.

The reference axis may be any physical structure that has a known orientation to the direction of action of action of gravity or orthogonal to the direction of action of gravity i.e. the physical structure is vertically orientated or horizontally orientated. Such objects may include door frames, window frames a treatment table.

The computer 13 computes the measurement and reference axes according to the measurement and reference points positioned on the image by the clinician or determined from the image by the computer 13. It should be noted that in each case the reference axis is non-parallel to the measurement axis in order that they intersect. The computer 13 determines a range of movement of a joint of the patient by computing an angle at the intersection of the measurement axis with the reference axis. If the measurement axis and the reference axis do not intersect, the measurement axis and/or the reference axis may be extrapolated.

It should be appreciated that the measurements may be stored in a database for analysis over time for patient monitoring. Successive measurements may be analysed by the clinician on the display 12 of the computer 13 to determine changes to the patient's range of movement. The successive measurements may be displayed to the clinician in a table or graphically to determine a trend. The trend may determine whether the patient's range of movement is improving or getting worse or otherwise.

In a second aspect of the invention, the camera 11 may include a tilt sensor, an accelerometer or any other device, in order to determine virtual reference axes. Horizontal and vertical virtual reference axes may be used as reference axes and superimposed on the image in order to determine a hip flexion, shoulder flexion and the like. The virtual reference axes are with respect to a direction of action of gravity i.e. vertical, or orthogonal to the direction of action of gravity i.e. horizontal.

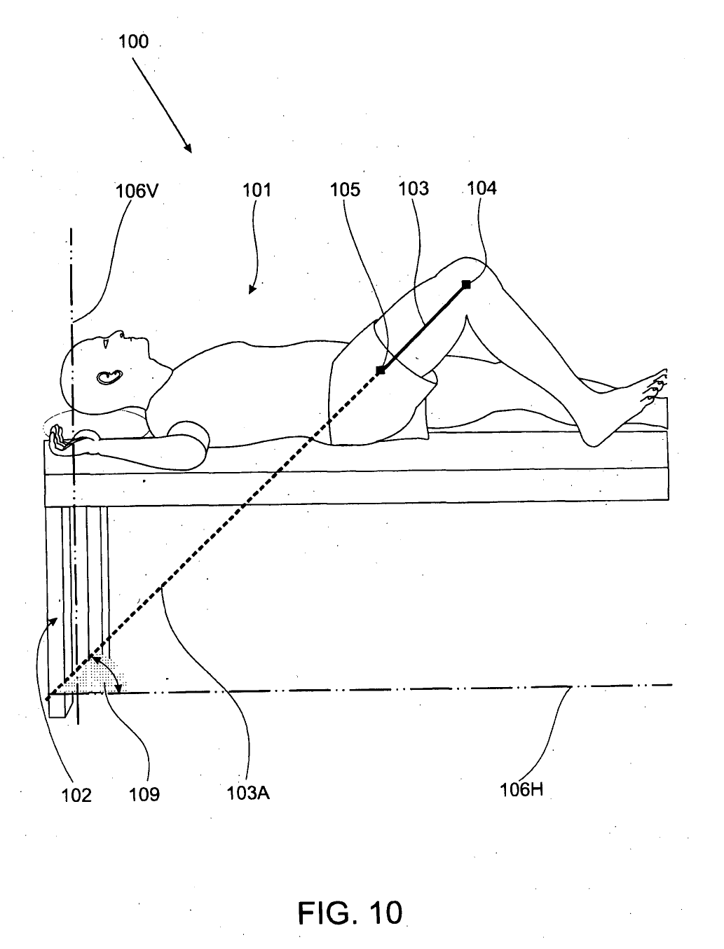

FIGS. 10 to 15 show measurements using virtual reference axes corresponding to FIGS. 4 to 9 respectively.

FIG. 10 shows an image 100 of a patient 101 on a treatment table 102 to determine a hip flexion of the patient 101 according to an embodiment of the present invention. Similarly to the embodiment of FIG. 4, measurement points 104, 105 are positioned on the patient's upper leg to determine a measurement axis 103. The measurement points 104, 105 are processed by the computer 13 to determine the measurement axis 103 which connects measurement points 104, 105. However in the present embodiment virtual reference axes 106H, 106V according to the camera's 11 accelerometer are used to determine a hip flexion. In one embodiment a virtual horizontal axis 106H and/or a virtual vertical axis 106V are determined according to the accelerometer in the camera 11. The axes 106H, 106V are then superimposed onto the image 100. Furthermore the measurement axis 103 may be extrapolated 103A to intersect with the virtual horizontal axis 106H and/or virtual vertical axis 106V to determine an angle 109 according to hip flexion. Although the virtual horizontal and virtual vertical axes 106H, 106V are shown at an edge of the image 100, it should be appreciated that the axes 106H, 106V may be positioned anywhere an image 100 and directly intersect with the axis of movement 103.

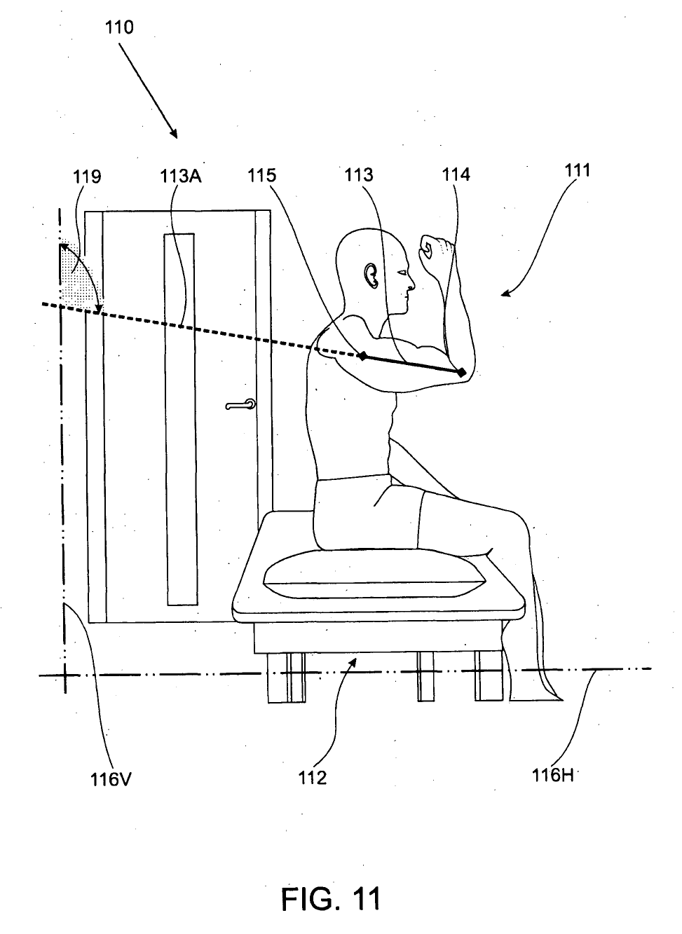

FIG. 11 shows an image 110 of a patient 111 on a treatment table 112 to determine a shoulder flexion of the patient 111 according to an embodiment of the present invention. Similarly to the embodiment shown in FIG. 5, a measurement axis 113 is determined by positioning measurement points 114, 115 on the patient's upper arm. The measurement points 114, 115 are processed by the computer 13 to determine the measurement axis 113 which connects measurement points 114, 115. An extrapolated measurement axis 113A intersects with a virtual vertical axis 116V to measure an angle 119 corresponding with the patient's 111 shoulder flexion. Again it should be appreciated that the virtual axes 116H, 116V may be positioned anywhere on the image 110. Furthermore, a reciprocal of the measured angle 119 may be computed in order to determine actual shoulder flexion.

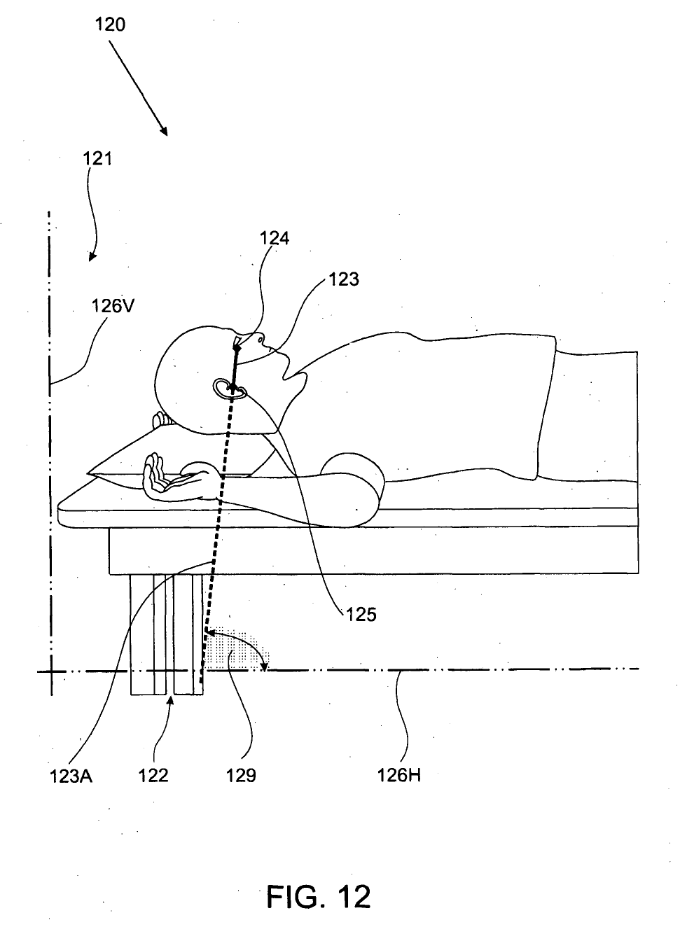

FIG. 12 shows an image 120 of a patient 121 on a treatment table 122 to determine a cervical flexion of the patient 121 according to an embodiment of the present invention. Similarly to the embodiment shown in FIG. 6, a measurement axis 123 is determined by positioning measurement points 124, 125 on the patient's 121 lateral eye canthus 124 and tragus 125. The measurement points 124, 125 are processed by the computer 13 to determine the measurement axis 123 which connects measurement points 124, 125. An extrapolated measurement axis 123A intersects with a virtual horizontal axis 126H to compute an angle 129 according to cervical flexion.

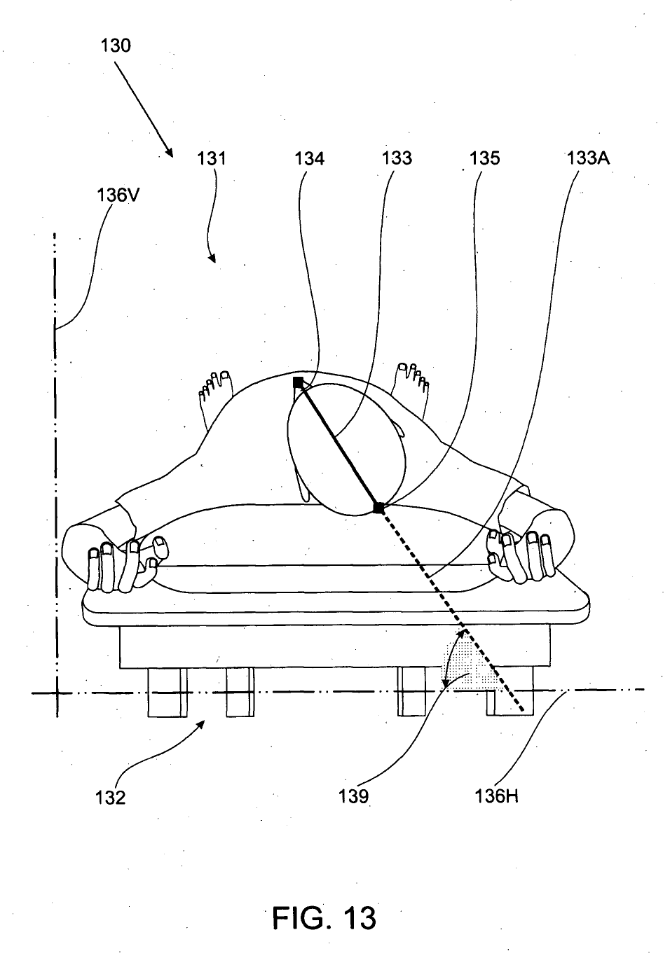

FIG. 13 shows an image 130 of a patient 131 on a treatment table 132 to determine a cervical rotation of the patient 131 according to an embodiment of the present invention. Similarly to the embodiment shown in FIG. 7, a measurement axis 133 is determined by positioning measurement points 134, 135 on the patient's 131 nose 134 and a point on the centre 135 of the back of the patient's 131 head. The measurement points 134, 135 are processed by the computer 13 to determine the measurement axis 133 which connects measurement points 134, 135. An extrapolated measurement axis 133A intersects with a virtual horizontal axis 136H to measure an angle 139 according to cervical rotation.

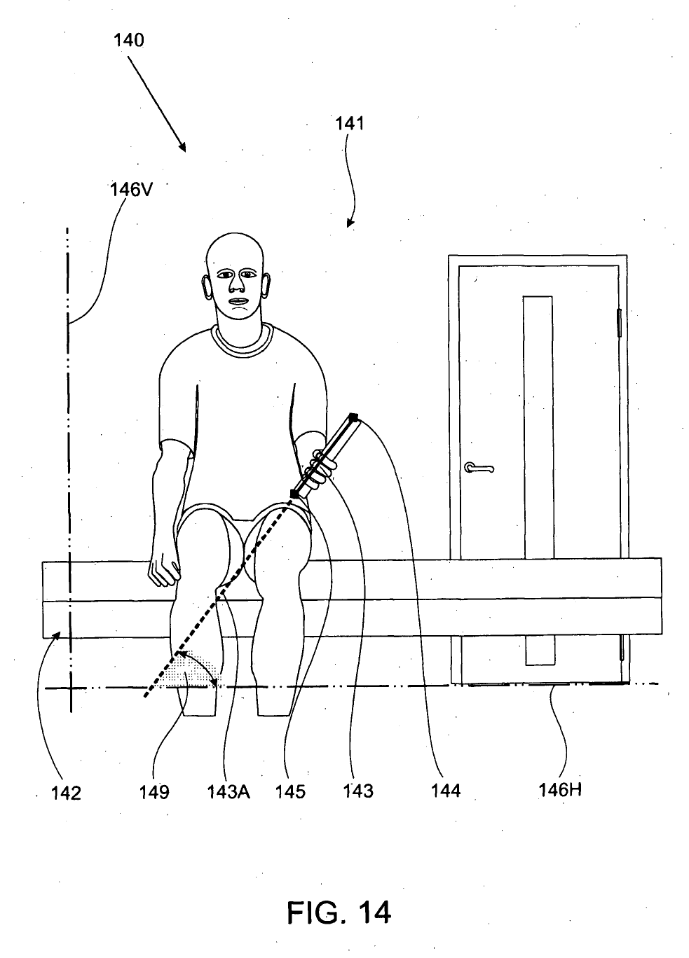

FIG. 14 shows an image 140 of a patient 141 on a treatment table 142 to determine a forearm supination of the patient 141 according to an embodiment of the present invention. Similarly to the embodiment shown in FIG. 8, a measurement axis 143 is determined by positioning measurement points 144, 145 on a baton held by the patient 141. The measurement points 144, 145 are processed by the computer 13 to determine the measurement axis 143 which connects measurement points 144, 145. An extrapolated measurement axis 143A intersects with a virtual horizontal axis 146H to compute an angle 149 corresponding with forearm supination.

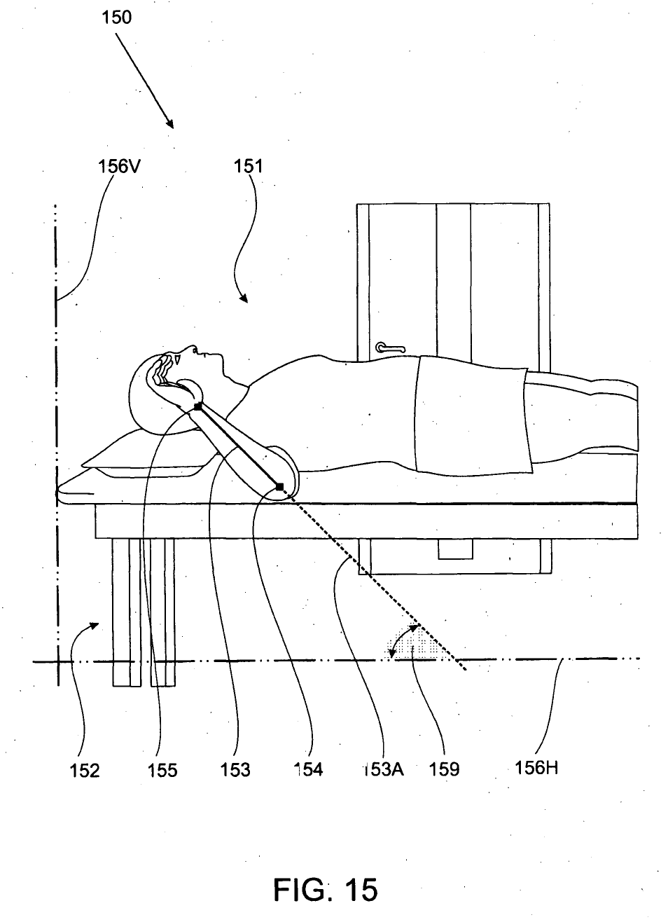

FIG. 15 shows an image 150 of a patient 151 on a treatment table 152 to determine a shoulder external rotation of the patient 151 according to an embodiment of the present invention. Similarly to the embodiment shown in FIG. 9, a measurement axis 153 is determined by positioning measurement points 154, 155 on a forearm of the patient 151. The measurement points 154, 155 are processed by the computer 13 to determine the measurement axis 153 which connects measurement points 154, 155. An extrapolated measurement axis 153A intersects with a virtual horizontal axis 156H to compute an angle 159 corresponding with shoulder external rotation.

Although in one embodiment the virtual reference axes are superimposed in the image, it should be appreciated that the virtual reference axes do not need to be displayed in the image as they are computer generated.

Although a limited number of examples of patient movements have been presented a person skilled in the art would appreciate that measurements of other patient postures and movements may be measured using the present invention.

The advantage of using virtual reference axes is that any error associated with determining a physical reference axis is removed. Furthermore, the measurements are not dependent on variations in reference axis at different locations.

In yet another aspect, the invention resides in a method of providing a scale for an image by projecting two or more parallel coherent light sources onto an object to create the scale and capturing an image of the object and the scale. The scale provides a known measurement in the image that may be used to compute distances in the image.

Using the scale, linear distances and displacements may be measured on an object or a patient in the image such as a foot length, head diameter and the like.

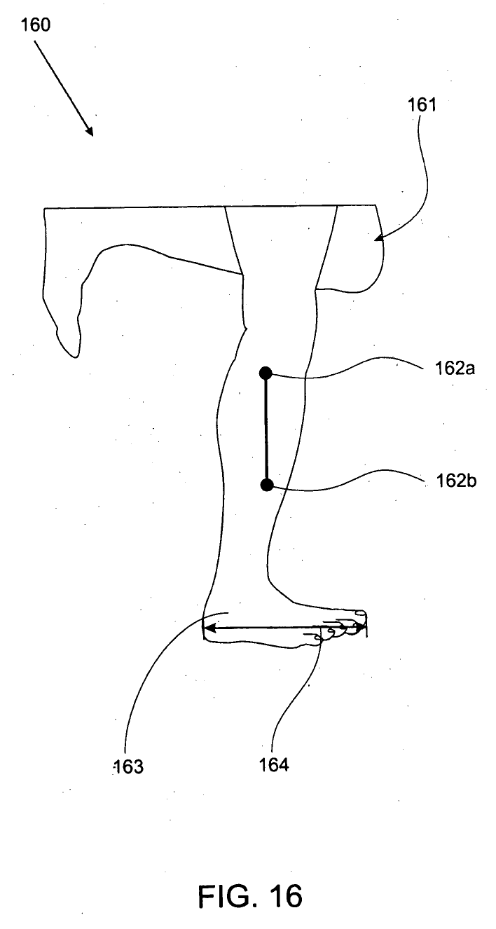

FIG. 16 shows an image 160 of a patient 161 to determine a foot length of the patient 161 according to an embodiment of a further aspect of the present invention. In this embodiment a scale is provided for the image 160 by projecting two parallel lasers on to a lower leg of the patient 161 and the image 160 is captured by the camera 11. Each laser produces a dot 162a, 162b on the lower leg of the patient 161. As the distance between the lasers is known and the lasers emit a coherent light source, the distance between the dots 162a, 162b in the image 160 is the same and may be used as a scale to compute linear distances and linear displacements from the image 160 that are approximately the same distance from the camera 11 as the dots 162a, 162b. In the present embodiment, a foot 163 measurement is computed by the computer 12. A marker 164 is placed or positioned on the image, defining the distance to be measured, and the computer computes the distance using the scale. In one embodiment, the image is analysed for the laser dots 162a, 162b to determine the number of pixels between them. The number of pixels between the laser dots 162a, 162b corresponds to a known distance which may be used as a scale to measure any other objects in the image.

It should be appreciated that any coherent electromagnetic source may also be used such as synchrotron radiation, infra-red, ultra-violet, microwave and radio frequency sources. Furthermore, it should be appreciated that a ruler or a fixed known length, such as a calibration sticker, may be placed on the object in the image as a scale. Although straight line distances are shown in the preferred embodiments, it should be appreciated that distances may be computed by the computer 12 that are curved or serpentine.

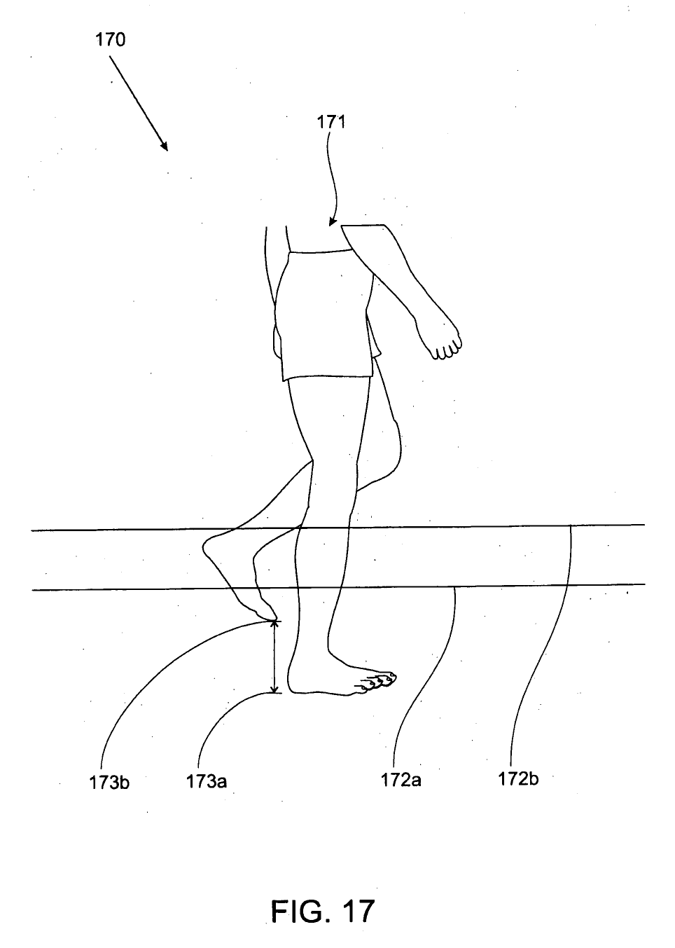

FIG. 17 shows an image 170 of a patient 171 to determine a height of a foot above the ground according to an embodiment of the present invention. In the present embodiment, a scale is added to the image 170 by projecting two parallel laser lines 172a, 172b, on to a lower leg of the patient 171. In order to produce the laser lines 172a, 172b, each laser is dithered as would be understood by a person skilled in the art. As the distance between the laser lines 172a, 172b is known, linear distances and displacements of objects and features in the image 170 may be computed by the computer 13 using the scale that are approximately the same distance from the camera 11 as the laser lines 172a, 172b. In the present embodiment a distance between a toe and the floor is measured by the clinician by positioning markers 173a, 173b on the image, and the distance between the markers 173a, 173b is computed using the scale.

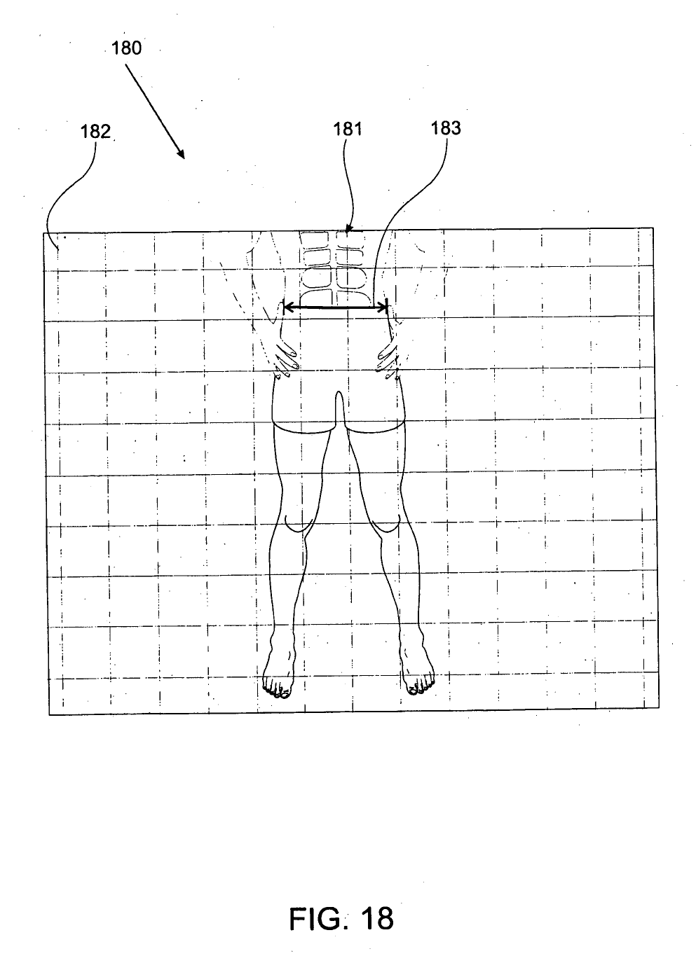

FIG. 18 shows an image 180 of a patient 181 including a scale in the form of a laser grid projected on to the image to compute a linear distance according to a further embodiment of the present invention. In the present embodiment, a plurality of parallel dithered lasers or any other coherent electromagnetic source may be used to superimpose a grid 182 onto the image 180 using the same principle as the embodiments detailed in FIGS. 15 and 16. Using the scale or grid 182, the computer 13 may compute linear distances or displacements in the image 180 that are approximately the same distance from the camera to the grid 182. In the present embodiment a width of a patient's waist 183 is measured.

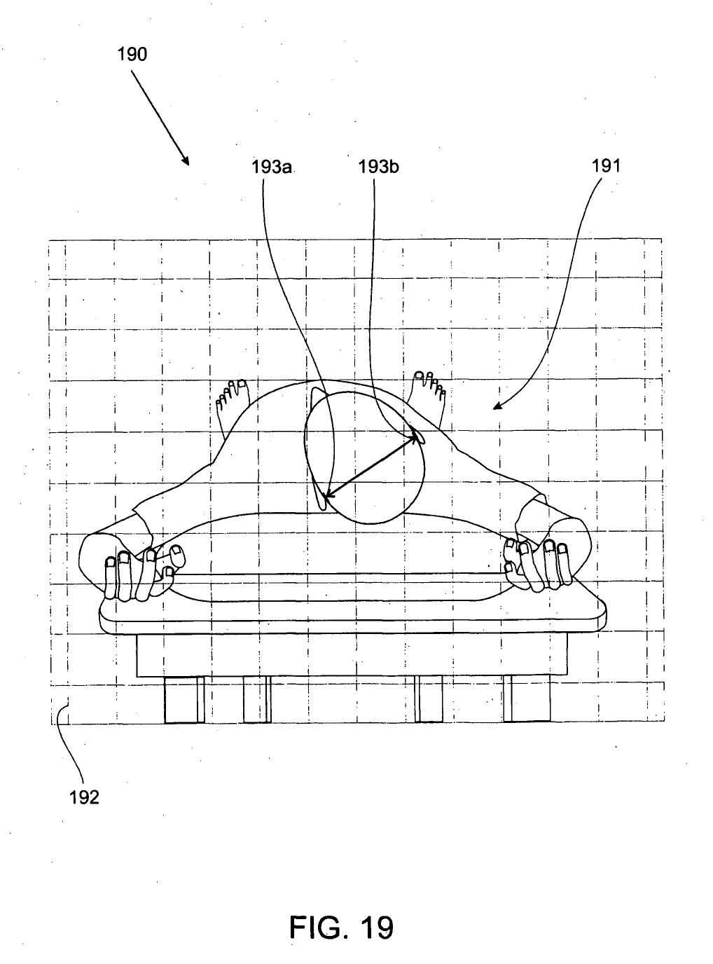

FIG. 19 shows an image 190 of a patient 191 using the grid of FIG. 18 to determine a head measurement according to a further embodiment of the present invention. In the present embodiment, a plurality of dithered parallel lasers may be used to project a grid 192 onto the image 190. Similarly, the grid 192 may be used as a scale in order to determine distances in the image. In the present example, the grid is used to calculate a width of the patient's 191 head. To make a measurement the clinician places or positions markers 193a, 193b on the image 190 and the computer 13 computes a distance according with the width of the patient's head using the scale or grid 192.

In a further embodiment of the invention, a distance between a point on a patient and the camera may be measured using a laser tape measure or any other, applicable device such as an ultrasonic tape measure. Once the distance to the patient has been measured the computer 13 computes a scale in the form of a grid and superimposes the grid onto the image according to the distance to the patient and the camera's field of view. To make a measurement the clinician places a marker on the image in a similar manner to FIG. 19. The grid may be a computer generated virtual grid determined from a gravity measuring device such as an accelerometer. Although in one embodiment the grid is shown on the image, it should be appreciated that the grid does not need to be displayed on the image in the case where the grid or scale is computer generated. One drawback of using the previous embodiments is that the camera 11 must be positioned perpendicular to the measurement axis on the patient in order to eliminate or reduce parallax errors. A further problem is that the patient may position themselves at an angle to the camera 11. In a third aspect of the invention, the parallax error may be reduced thus positioning of the patient and the camera 11 is not as critical.

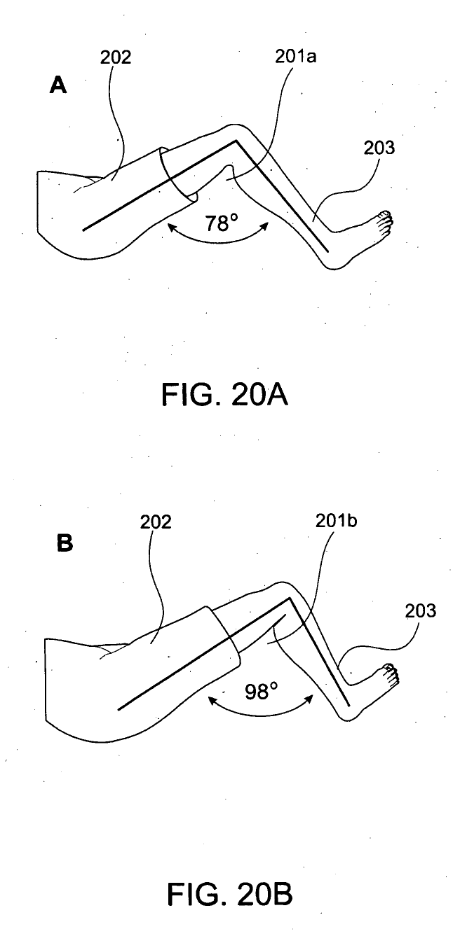

FIG. 20 shows an image outlining the problem associated with parallax errors. FIG. 20A shows an angle 201a measured between an upper leg 202 and a lower leg 203 where the patient is perpendicular to the camera's field of view. FIG. 20B shows an angle 201b where the patient is positioned at an angle, and not perpendicular to the camera. As shown the computed angle 201 is different between each camera angle of FIG. 20A and FIG. 20B.

The parallax error may be compensated for by using a parallax algorithm. The parallax algorithm is executed by the computer 13 and corrects an image by moving pixels in the image. Once corrected, the image appears as if the camera was positioned substantially perpendicular to a patient in the image. The parallax algorithm corrects the image according to virtual reference axes, and/or identifying physical reference axes in the images such as door frames, window frames and the like.

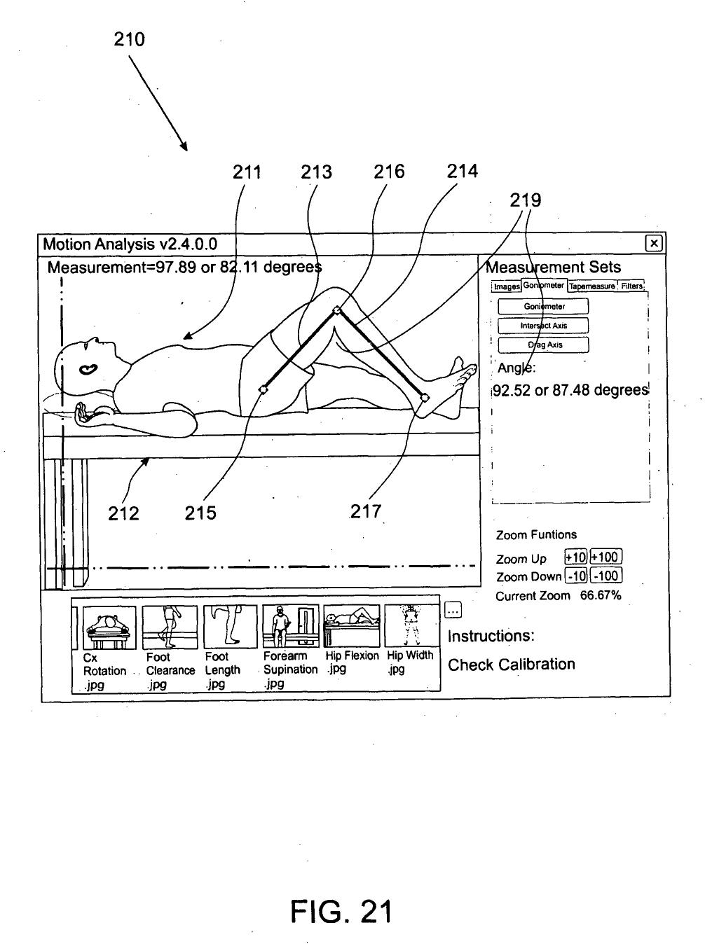

FIG. 21 shows a screen shot 210 of a measurement of an angle between a patient's upper leg and lower leg according to an embodiment of the present invention. In this example, a camera is positioned perpendicular to the patient 211. As such there is minimal parallax error and a horizontal plinth of a treatment table 212 is substantially parallel with a horizontal virtual reference axis. A first measurement axis 213 and a second measurement axis 214 may be determined by placing measurement points 215, 216, 217 according to bony landmarks 215, 216, 217 of the patient 212 as previously described. In this example the bony landmarks 215, 216, 217 accord to a point along a shaft of the femur 215, a Lateral condyle of the knee 216 and an ankle 217 of the patient 211. The computer 13 may then compute an actual angle 219 at an intersection of the first measurement axis 213 and the second measurement axis 214. In this example the actual angle 219 is computed by the computer 13 to be 92.52 degrees.

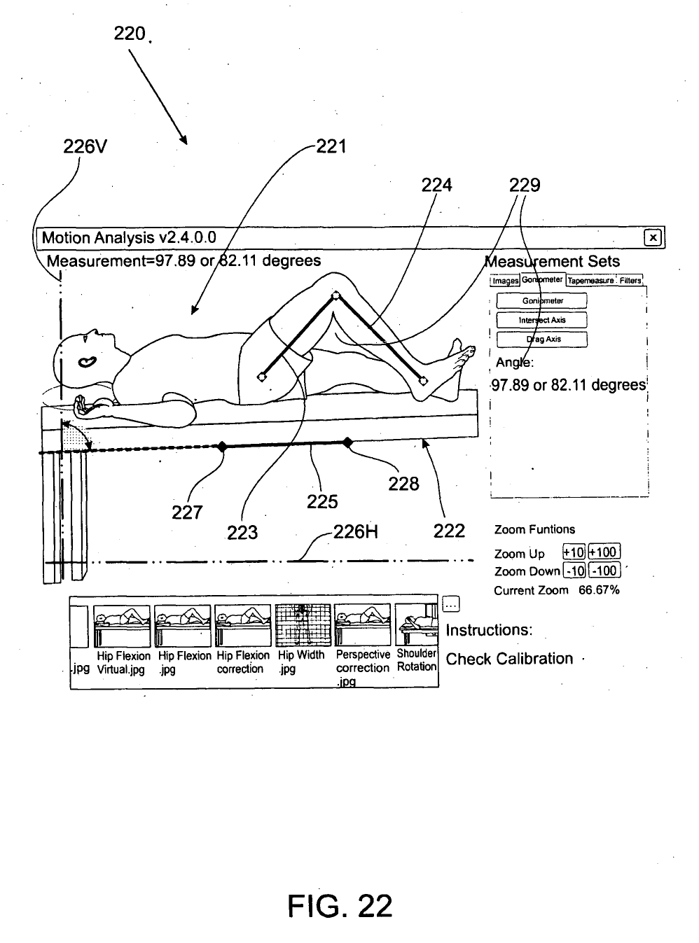

FIG. 22 shows a screen shot 220 of the image of FIG. 21 where a camera is not perpendicular to a patient 221 according to an embodiment of the present invention. As the camera is not perpendicular with a side of the patient 221, a horizontal plinth of the treatment table 222 is not parallel with a horizontal virtual reference axis 226H. Thus an angle formed between the horizontal plinth and the vertical reference axis 226V is not ninety degrees. Similarly to FIG. 21, a first measurement axis 223 and a second measurement axis 224 are determined from bony landmarks identified from the image. Due to a parallax error an angle 229 at an intersection of the first measurement axis 223 and the second measurement axis 224 is computed by the computer 13 to be 97.89 degrees.

To correct the parallax error, virtual reference axes 226H, 226V are superimposed onto the image according to an accelerometer (or any other suitable gravity measuring device). The computer 13 using a parallax algorithm according to the virtual reference axes 226H, 226V and at least one physical reference axis 225, modifies the image by moving pixels in the image. The clinician indicates on the image that the horizontal plinth of the treatment table should be horizontal by placing markers 227, 228 on the image in order to determine the physical reference axis 225. Again, although in one embodiment the virtual reference axes are shown on the image, it should be appreciated that the virtual reference axes do not need to be displayed on the image as they are computer generated.

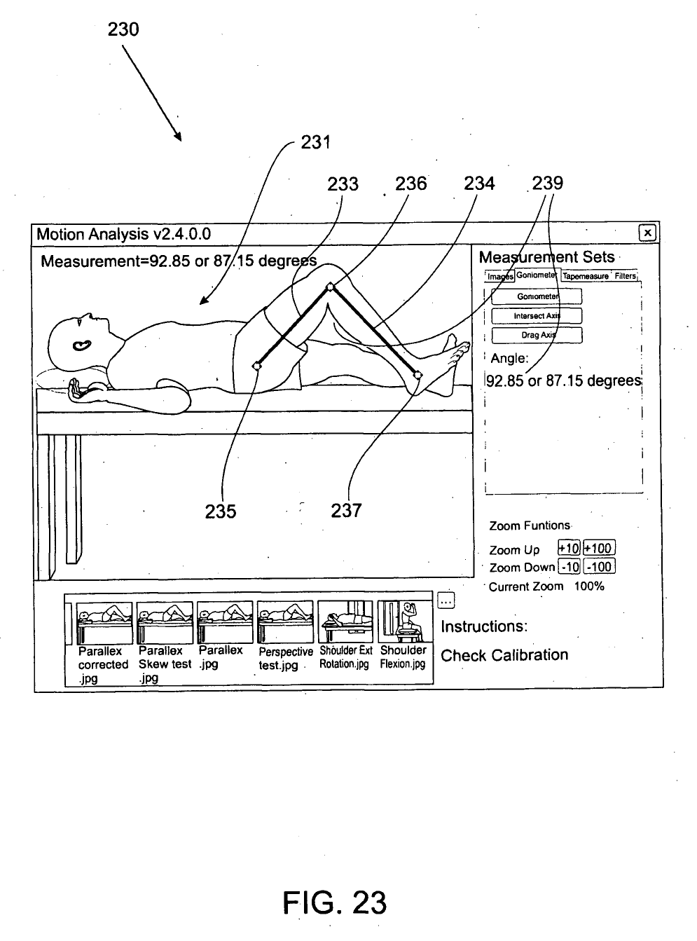

FIG. 23 shows a screen shot 230 of a corrected image of FIG. 22 according to an embodiment of the present invention. In this embodiment a parallax algorithm has been applied to the image shown in FIG. 22 according to at least one virtual reference axis and at least one physical reference axis. The clinician places measurement points 235, 236, 237 on the corrected image in order to determine a first measurement axis 233 and a second measurement axis 234. The computer may then compute a corrected measurement 239 at an intersection of the first measurement axis 233 and the second measurement axis 234. In the present embodiment, the corrected angle 239 is computed to be 92.85 which is extremely close to the actual measurement of 92.52 degrees.

The present invention has many advantages over the prior art including:
1) Physical dimensions, static postures or movement measurements may be made more accurately;
2) Physical dimensions, static postures or movement measurements may be carried out remotely or the patient may perform self measurements;
3) The present invention allows both angular and length measurements to be taken;
4) The present invention allows measurements to be stored and historically analysed; and
5) The present invention may also adjust the measurement to compensate for a parallax error.

The above description of various embodiments of the present invention is provided for purposes of description to one of ordinary skill in the related art. It is not intended to be exhaustive or to limit the invention to a single disclosed embodiment. As mentioned above, numerous alternatives and variations to the present invention will be apparent to those skilled in the art of the above teaching. For example the process of determining the reference axis may be automatically determined by an algorithm that automatically determines measurement axis of the patient or physical reference axes. Accordingly, while some alternative embodiments have been discussed specifically, other embodiments will be apparent or relatively easily developed by those of ordinary skill in the art. Accordingly, this invention is intended to embrace all alternatives, modifications and variations of the present invention that have been discussed herein, and other embodiments that fall within the spirit and scope of the above described invention.

1. A method of measuring a range of movement of a joint of a patient including the steps of:
capturing an image including at least part of the patient;
processing measurement points positioned on the image;
determining a measurement axis that connects the measurement points;
determining a reference axis; and
measuring an angle at an intersection of the measurement axis and the reference axis according to the range of movement of the joint of the patient.

2. The method of claim 1 including the step of displaying the angle on a display of a computer.

3. The method of claim 1 wherein at least one of the measurement axis and the reference axis are extrapolated such that the measurement axis and the reference axis intersect.

4. The method of claim 1 wherein the angle is recorded in a database on a computer with respect to time.

5. The method of claim 4 wherein angles recorded in the database are displayed to the clinician on the screen of the computer to determine changes in the angle.

6. The method of claim 1 wherein the reference axis is a physical reference axis.

7. The method of claim 1 wherein the reference axis is a computer generated virtual reference axis.

8. The method of claim 7 wherein the virtual reference axis is determined with respect to vertical.

9. The method of claim 8 wherein the virtual reference axis is determined using an accelerometer.

10. The method of claim 1 wherein the image is captured from a photo.

11. The method of claim 1 wherein the image is captured from a video.

12. The method of claim 1 wherein the image is corrected for a parallax error.

13. The method of claim 12 wherein the parallax error is corrected using an algorithm based on the physical reference axis and the virtual reference axis and moves pixels in the image to create a corrected image.

14. A method of measuring a distance or displacement in an image including the steps of:
providing a scale for the image;
capturing the image including at least part of a patient and the scale;
positioning a marker on the image that defines a distance to be measured; and
measuring the distance using the marker and the scale.

15. The method of claim 14 wherein the scale is provided by two or more parallel coherent electromagnetic sources projected onto the patient wherein the distance is known between the two parallel coherent electromagnetic sources.

16. The method of claim 15 wherein the two parallel coherent electromagnetic sources are lasers.

17. The method of claim 15 wherein the two or more parallel coherent light sources form two or more parallel lines on the image.

18. The method of claim 14 wherein the scale is computer generated.

19. A system for determining a range of movement of a joint of a patient including;
a camera; and
a computer connected to the camera, the computer including
a processor; and
a memory coupled to the processor, wherein the memory includes computer readable program code components configured to cause:
capturing an image including at least part of the patient;
processing measurement points positioned on the image;
determining a measurement axis that connects the measurement points;
determining a reference axis; and
measuring an angle at an intersection of the measurement axis and the reference axis according to the range of movement of the joint of the patient.

20. The system of claim 19 wherein the angle is recorded in a database on the computer with respect to time.
